(19) United States
(12) Patent Application Publication
(10) Pub. No.: US 2026/0224905 A1
(43) Pub. Date: Aug. 6, 2026

(54) MEDICAL DEVICE ELECTRODE, COATINGS AND PROCESSES

(71) Applicant: Medtronic, Inc., Minneapolis, MN (US)

(72) Inventors: Zhongping Yang, Woodbury, MN (US); Thomas A. Anderson, New Hope, MN (US)

(21) Appl. No.: 19/150,409

(22) PCT Filed: Jan. 9, 2024
(86) PCT No.: PCT/IB2024/050211

§ 371 Date: Jul. 23, 2025

Related U.S. Application Data
(60) Provisional application No. 63/481,674, filed on Jan. 26, 2023.

(51) Int. Cl. A61N 1/375

(52) U.S. Cl. CPC A61N 1/3756; A61N 1/37512

(57) ABSTRACT

A device includes an electrode, a power source, electrical pacing circuitry, and a coating applied to one or more surfaces of the electrode. The electrical pacing circuitry is coupled to the power source and configured to deliver cardiac pacing. The coating comprises a first adhesive layer comprising porous titanium nitride and a second electrically insulated layer comprising parylene. A thickness of the second electrically insulated layer is substantially uniform throughout the entirety of the coating. A method to apply the coating includes applying the first adhesive layer to one or more surfaces of the electrode at a first time, applying a plasma cleaning process to the first adhesive layer at a second time that is after the first time, and applying the second electrically insulated layer to one or more surfaces of the electrode coated with the first adhesive layer at a third time that is after the second time.

This application claims the benefit of U.S. Provisional Patent Application Ser. No. 63/481,674, filed Jan. 26, 2023, the entire content of which is incorporated herein by reference.

### TECHNICAL FIELD

The present application relates to electrodes of implantable medical devices, and more particularly to techniques for forming coated electrodes.

### BACKGROUND

An example implantable medical device is a leadless pacing device that may assist cardiac function in a patient. A leadless pacing device may offer advantages over a conventional pacing device, as the absence of leads may allow for fewer complications and improved patient outcomes. To ensure long-term biostability, reliability, and performance, the pacing electrode of the leadless pacing device may be insulated with parylene (polyparaxylylene), a flexible, biocompatible polymer that may help reduce adverse reactions to the implanted pacing device. Electrodes for other cardiac pacing devices, or other electrical stimulation or sensing devices, such as electrodes carried by implantable leads, may also be insulated with parylene. An uninsulated portion of the electrode may be defined to act as the electrically active portion for sensing and/or stimulation as well as be defined to provide desired sensing and/or stimulation performance.

### SUMMARY

Parylene deformation in the form of delamination, swelling, or tearing may compromise the long-term biostability and function of the implanted device. For example, parylene deformation may negatively affect the sensing and/or stimulation performance of the implanted device. Some cardiac pacing devices, such as leadless pacing devices, include an elongate electrode that extends from a first heart chamber to myocardial tissue of a second heart chamber. The distal end portion of such an electrode may be uninsulated to allow sensing electrical activity of the second heart chamber and pacing of the second heart chamber, with the insulation on the remainder of the electrode impeding sensing and/or pacing of the first heart chamber by the electrode. Parylene deformation may allow undesired sensing and/or stimulation of the first heart chamber by the elongate electrode, negatively affecting the sensing and therapy performance of such implanted devices.

According to the techniques of this disclosure, a coating may be applied to one or more surfaces of an electrode, wherein the coating includes a first adhesive layer comprising an adhesive substrate, such as porous titanium nitride, and a second electrically insulated layer comprising parylene. The porous titanium nitride layer may promote adhesion of the parylene layer onto the one or more surfaces of the electrode, which may reduce failure of the implantable medical device and increase its long-term biostability and reliability. In some examples, the electrode is elongate, e.g., with the parylene coating all but a distal portion of the electrode. In some examples, the electrode is configured as a coil. An electrode configured according to the techniques of this disclosure may have a significantly reduced likelihood of insulation deformation, thereby advantageously ensuring desired performance of the medical device.

In some examples, the disclosure describes an implantable medical device that includes an electrode, a power source, electrical pacing circuitry, and a coating applied to one or more surfaces of the electrode. The electrical pacing circuitry is coupled to the power source and configured to deliver cardiac pacing therapy. The coating applied to one or more surfaces of the electrode comprises a first adhesive layer comprising porous titanium nitride and a second electrically insulated layer comprising parylene. A thickness of the second electrically insulated layer is substantially uniform throughout the entirety of the coating.

In some examples, the disclosure describes a method for forming an implantable medical device that includes an electrode, a power source, electrical pacing circuitry that is coupled to the power source and that is configured to deliver cardiac pacing therapy, and a coating applied to one or more surfaces of the electrode, wherein the coating comprises a first adhesive layer comprising porous titanium nitride and a second electrically insulated layer comprising parylene. The method includes applying the first adhesive layer comprising porous titanium nitride to the one or more surfaces of the electrode at a first time. The method also includes applying a plasma cleaning process to the first adhesive layer at a second time that is after the first time. The method also includes applying the second electrically insulated layer comprising parylene to the one or more surfaces of the electrode coated with the first adhesive layer at a third time that is after the second time.

In some examples, the disclosure describes an implantable medical device that includes a power source, electrical pacing circuitry, a first electrode, a second electrode, and a coating. The electrical pacing circuitry is coupled to the power source and configured to deliver cardiac pacing therapy. The first electrode is configured to extend distally from a distal portion of the implantable medical device. The second electrode is located on the distal portion of the implantable medical device. The coating comprises a first adhesive layer comprising porous titanium nitride and a second electrically insulated layer comprising parylene.

This summary is intended to provide an overview of the subject matter described in this disclosure. It is not intended to provide an exclusive or exhaustive explanation of the techniques as described in detail within the accompanying drawings and description below. Further details of one or more examples are set forth in the accompanying drawings and the description below. Other features, objects, and advantages will be apparent from the description and drawings, and from the statements provided below.

### BRIEF DESCRIPTION OF THE DRAWINGS

FIG. 1 is a conceptual drawing illustrating an example device implanted in the heart of a patient, in accordance with one or more aspects of this disclosure.

FIG. 2 is a functional block diagram illustrating an example configuration of the IMD of FIG. 1, in accordance with one or more aspects of this disclosure.

FIG. 3 is a conceptual diagram of the device of FIGS. 1 and 2 implanted at a target implant site.

FIG. 4 is a conceptual diagram illustrating an example implantable medical device including an electrode.

FIG. 5 is a conceptual diagram illustrating an example coating applied to one or more surfaces of an electrode.

FIG. 6 is a flow diagram illustrating an example technique for forming an implantable medical device including a coated electrode.

### DETAILED DESCRIPTION

The disclosure describes implantable medical devices including an electrode coated with a first adhesive layer comprising porous titanium nitride and a second electrically insulated layer comprising parylene and methods for forming such implantable medical devices. In some examples, the implantable medical device is a leadless pacing device, but in other examples may be other devices such as other leadless stimulation devices or implantable leads. In some examples, the electrode is a pacing (or more generally stimulation) electrode coil or helix. In some examples, the electrode includes a plurality of turns of at least one filar (e.g., a conductive metal wire) and delivers electrical stimulation (e.g., pacing stimulation, such as antitachycardia pacing, bradycardia pacing, and/or post-shock pacing, or the like) to a patient. In some examples, one or more surfaces of the electrode are coated with a parylene layer to improve the long-term biostability, reliability, and performance of the implantable medical device. In some examples, one or more surfaces of the electrode are coated with a first adhesive layer comprising porous titanium nitride to improve adhesion of the parylene layer.

For example, a first adhesive layer comprising porous titanium nitride is applied to one or more surfaces of an electrode at a first time. A second electrically insulated layer comprising parylene is then applied to the one or more surfaces of the electrode coated with the first adhesive layer at a second time that is after the first time. In some examples, after applying the porous titanium nitride layer and prior to applying the parylene layer, a plasma cleaning process may be applied to the porous titanium nitride layer to remove any surface impurities or contaminants. In some examples, the first adhesive layer comprising porous titanium nitride is thermally bonded to the second electrically insulated layer comprising parylene. In some examples, the first adhesive layer may cover a first amount, e.g., all surfaces of the electrode, while the second electrically insulated layer may cover a second amount less than the first amount, thus leaving a portion of the first adhesive layer exposed. In some examples, the first and second amounts may be coextensive, thus leaving a portion of bare electrode exposed. In some examples, the exposed portions may be a distal tip of the electrode.

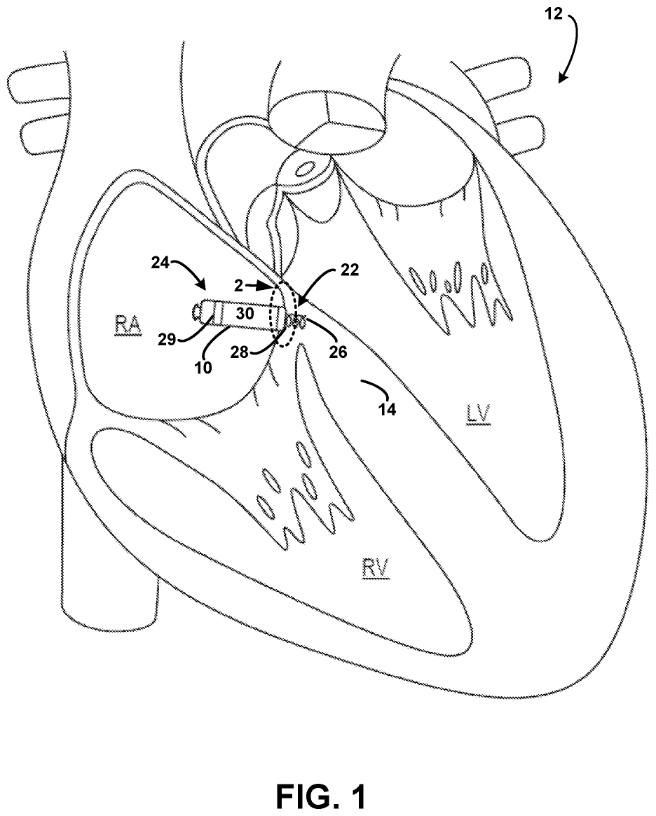

FIG. 1 is a conceptual drawing illustrating an example device 10 implanted in the heart 12 of a patient, in accordance with one or more aspects of this disclosure. Device 10 is shown implanted in the right atrium (RA) of the patient's heart 12 in a target implant region 2, such as triangle of Koch, in heart 12 of the patient with a distal end of device 10 directed toward the left ventricle (LV) of the patient's heart 12. Although in the example of FIG. 1 the distal end of device 10 is directed toward the LV, the distal end may be directed to other targets, such as interventricular septum of heart 12, in some examples. Target implant region 2 may lie between the bundle of His and the coronary sinus and may be adjacent the tricuspid valve.

Device 10 includes housing 30. Device 10 includes a distal end 22 and a proximal end 24. Distal end 22 includes a first electrode 26 and a second electrode 28, both of which may extend distally from housing 30. First electrode 26 extends from distal end 22 and may penetrate through the wall tissue of a first chamber (e.g., the RA in the illustrated example) into wall tissue of a second chamber (e.g., the LV in the illustrated example). Second electrode 28 extends from distal end 22 and is configured to flexibly maintain contact with the wall tissue of the first chamber without penetration of the wall tissue of the first chamber by the second electrode. Device 10 may also include a third electrode 29, which may be formed as an uninsulated portion of housing 30, and used as a reference electrode paired with either or both of first electrode 26 and second electrode 28 for sensing and stimulation.

The configuration of electrodes 26 and 28 illustrated in FIG. 1 allows device 10 to sense cardiac signals and/or deliver cardiac pacing to multiple chambers of heart 12, e.g., the RA and ventricles in the illustrated example. In this manner, the configuration of electrodes 26 and 28 may facilitate the delivery of A-V synchronous pacing by single device 10 implanted within the single chamber, e.g., the RA. While device 10 is implanted at target implant region 2 to sense in and/or pace the RA and ventricles in the example shown in FIG. 1, a device having an electrode configuration in accordance with the examples of this disclosure may be implanted at any of a variety of locations to sense in and/or pace any two or more chambers of heart 12. For example, device 10 may be implanted at region 2 or another region, and first electrode 26 may extend into tissue, e.g., myocardial tissue, of the LV or interventricular septum to, for example, facilitate the delivery of A-V synchronous pacing. Furthermore, a device having an electrode configuration in accordance with the examples of this disclosure may be implanted at any of a variety of locations within a patient for sensing and/or delivery of therapy to other patient tissue.

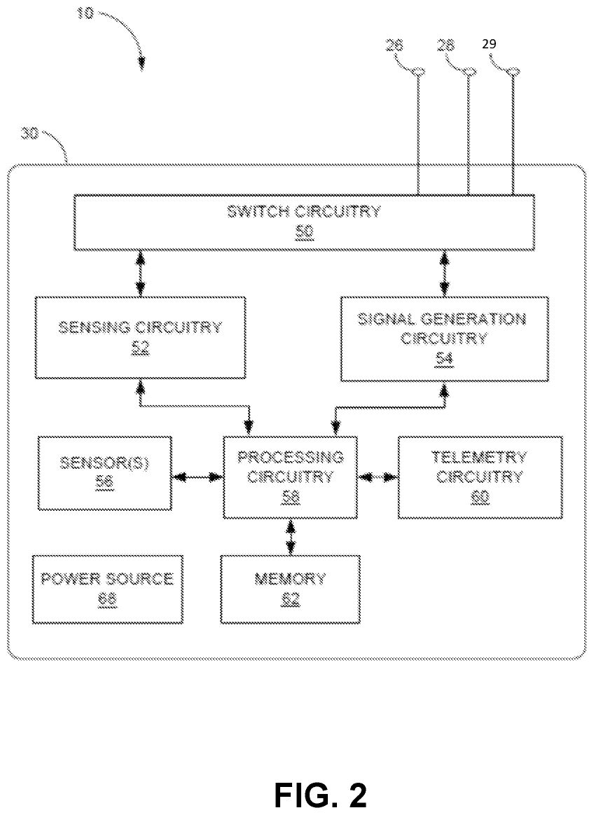

FIG. 2 is a functional block diagram illustrating an example configuration of device 10 that includes housing 30. As illustrated in FIG. 1, device 10 include electrodes 26, 28, and 29, which may be configured as described with respect to FIG. 1. For example, as described with respect to FIG. 1, first electrode 26 may be configured to extend from distal end 22 of housing 30 and may penetrate through the wall tissue of a first chamber (e.g., the RA) into wall tissue of a second chamber (e.g., the LV). Second electrode 28 extends from distal end 22 of housing 30 and is configured to flexibly maintain contact with the wall tissue of the first chamber without penetration of the wall tissue of the first chamber by the second electrode.

In the example shown in FIG. 2, device 10 includes switch circuitry 50, sensing circuitry 52, signal generation circuitry 54, sensor(s) 56, processing circuitry 58, telemetry circuitry 60, memory 62, and power source 68. The various circuitry may be, or include, programmable or fixed function circuitry configured to perform the functions attributed to respective circuitry. Memory 62 may store computer-readable instructions that, when executed by processing circuitry 58, cause device 10 to perform various functions. Memory 62 may be a storage device or other non-transitory medium. The components of device 10 illustrated in FIG. 2 may be housed within housing 30.

Signal generation circuitry 54 generates electrical stimulation signals, e.g., cardiac pacing pulses. Switch circuitry 50 is coupled to electrodes 26, 28, and 29 and may include one or more switch arrays, one or more multiplexers, one or more switches (e.g., a switch matrix or other collection of switches), one or more transistors, or other electrical circuitry. Switch circuitry 50 is configured to direct stimulation signals from signal generation circuitry 54 to a selected combination of electrodes 26, 28 and 29 having selected polarities, e.g., to selectively deliver pacing pulses to the RA, ventricles, or interventricular septum of heart 12. For example, in order to pace one or both of the ventricles, switch circuitry 50 may couple first electrode 26, which has penetrated to wall tissue of a ventricle or the intraventricular septum, to signal generation circuitry 54 as a cathode, and third electrode 29 to signal generation circuitry 54 as an anode. As another example, in order to pace the RA, switch circuitry 50 may couple second electrode 28, which flexibly maintains contact with the RA endocardium, to signal generation circuitry 54 as a cathode, and third electrode 29 to signal generation circuitry 54 as an anode.

Switch circuitry 50 may also selectively couple sensing circuitry 52 to selected combinations of electrodes 26, 28, and 29, e.g., to selectively sense the electrical activity of either the RA or ventricles of heart 12. Sensing circuitry 52 may include filters, amplifiers, analog-to-digital converters, or other circuitry configured to sense cardiac electrical signals via electrodes 26 and 28. For example, switch circuitry 50 may couple each of first electrode 26 and second electrode 28 in combination with third electrode 29 to respective sensing channels provided by sensing circuitry 52 to respectively sense either ventricular or atrial cardiac electrical signals. In some examples, sensing circuitry 52 is configured to detect events, e.g., depolarizations, within the cardiac electrical signals, and provide indications thereof to processing circuitry 58. In this manner, processing circuitry 58 may determine the timing of atrial and ventricular depolarizations, and control the delivery of cardiac pacing, e.g., AV synchronized cardiac pacing, based thereon. Processing circuitry 58 may include any one or more of a microprocessor, a controller, a digital signal processor (DSP), an application specific integrated circuit (ASIC), a field-programmable gate array (FPGA), discrete logic circuitry, or any other processing circuitry configured to provide the functions attributed to processing circuitry 58 herein may be embodied as firmware, hardware, software or any combination thereof.

Sensor(s) 56 may include one or more sensing elements that transduce patient physiological activity to an electrical signal to sense values of a respective patient parameter. Sensor(s) 56 may include one or more accelerometers, optical sensors, chemical sensors, temperature sensors, pressure sensors, or any other types of sensors. Sensor(s) 56 may output patient parameter values that may be used as feedback to control sensing and delivery of therapy by device 10.

Telemetry circuitry 60 supports wireless communication between device 10 and an external programmer (not shown in FIG. 2) or another computing device under the control of processing circuitry 58. Processing circuitry 58 of device 10 may receive, as updates to operational parameters from the computing device, and provide collected data, e.g., sensed heart activity or other patient parameters, via telemetry circuitry 60. Telemetry circuitry 60 may accomplish communication by radiofrequency (RF) communication techniques, e.g., via an antenna (not shown).

Power source 68 delivers operating power to various components of device 10. Power source 68 may include a rechargeable or non-rechargeable battery and a power generation circuit to produce the operating power. Recharging may be accomplished through proximal inductive interaction between an external charger and an inductive charging coil within device 10.

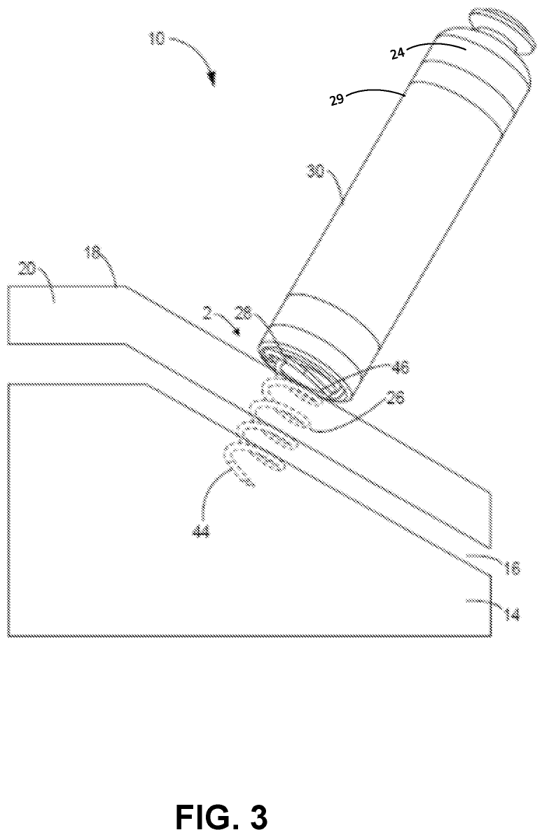

FIG. 3 is a conceptual diagram of device 10 implanted at target implant region 2. First electrode 26 may be inserted such that tissue becomes engaged with the helix of first electrode 26. As first electrode 26 becomes engaged with tissue, first electrode 26 pierces into the tissue at target implant region 2 and advances through atrial myocardium 20 and central fibrous body 16 to position first electrically active region 44 in ventricular myocardium 14 as shown in FIG. 3. In some examples, first electrode 26 penetrates into the interventricular septum. In some examples, first electrode 26 does not perforate entirely through the ventricular endocardial or epicardial surface.

In some examples, manual pressure applied to the housing proximal end 24, e.g., via an advancement tool, provides the longitudinal force to pierce the cardiac tissue at target implant region 2. In some examples, actuation of an advancement tool rotates device 10 and first electrode 26 configured as a helix about a longitudinal axis. The rotation of the helix about the longitudinal axis advances first electrode 26 through atrial myocardium 20 and central fibrous body 16 to position first electrically active region 44 in ventricular myocardium 14 as shown in FIG. 3.

As first electrode 26 advances into the tissue, the distance between second electrode 28 and atrial endocardium 18 decreases until second electrode 28 contacts, and may press against, the surface of atrial endocardium 18 so that heart tissue becomes engaged with second electrically active region 46. Second electrode 28 is held in contact with atrial endocardium 18 by first electrode 26, e.g., retraction of second electrode 28 from the surface of atrial endocardium 18 is prevented by first electrode 26. Second electrode 28 is also configured, as described herein, to flexibly maintain contact with atrial endocardium 18. In some examples, second electrode is elastically deformable toward distal end 22 (FIG. 1) of housing 30, and has a spring bias urging second electrode distally from distal end 22. First electrode 26 can be the sole fixation feature of device 10 in some examples. The distance first electrode 26 extends from housing 30 can be selected so first electrically active region 44 reaches an appropriate depth in the tissue layers to reach the targeted pacing and sensing site, in this case in ventricular myocardium 14, without puncturing all the way through into an adjacent cardiac chamber.

Target implant region 2 in some pacing applications is along atrial endocardium 18, substantially inferior to the AV node and bundle of His. First electrode 26 can have a length that penetrates through atrial endocardium 18 in target implant region 2, through the central fibrous body 16 and into ventricular myocardium 14 without perforating through the ventricular endocardial surface. In some examples, when the full length of first electrode 26 is fully advanced into target implant region 2, first electrically active region 44 rests within ventricular myocardium 14 and second electrode 28 is positioned in intimate contact with atrial endocardium 18. First electrode 26 may extend from distal end 22 of housing 30 approximately 3 mm to 12 mm in various examples. In some examples, first electrode 26 may extend a distance from housing 30 of at least 3 millimeters (mm), at least 3 mm but less than 20 mm, less than 15 mm, less than 10 mm, or less than 8 mm in various examples. The diameter of first and second electrodes 26 and 28 may be less than 2 mm and may be 1 mm or less, or even 0.6 mm or less.

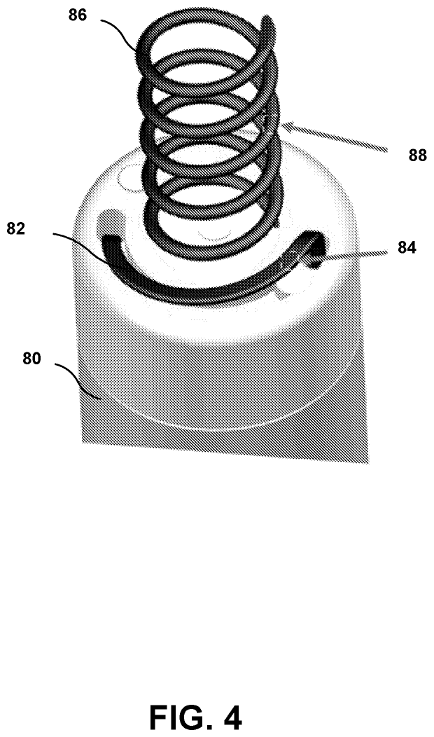

FIG. 4 is a conceptual diagram illustrating an example implantable medical device (IMD) 80 including an elongate electrode, which can be in the form of an elongate electrode coil 86 or helix extending distally from a distal portion of IMD 80. IMD 80 may be substantially similar to device 10 of FIGS. 1, 2, and 3. Instead of the depicted elongate electrode coil 86, the elongate electrode can comprise an elongate shaft, e.g., a wholly or substantially straight shaft, needle or dart extending distally from a distal portion of IMD 80, along or parallel to a central longitudinal axis of IMD 80. Such an elongate shaft electrode can be curved along all or a portion of its length, and/or can be oriented at an oblique angle with respect to the central longitudinal axis along all or a portion of its length, and/or include a sharp or tissue penetrating distal tip. Alternatively, elongate electrode coil 86 may be substantially similar to first electrode 26 of FIGS. 1, 2, and 3. As shown in the example of FIG. 4, elongate electrode coil 86 includes one or more surfaces such as elongate electrode coil surface(s) 88. In the example of FIG. 4, IMD 80 also includes a shorter electrode, which can be in the form of a partial electrode coil 82 or partial helix positioned on a distal end of IMD 80. Instead of the depicted partial electrode coil 82, the shorter electrode can comprise any other suitable electrode or electrode surface, such as a circular “button” electrode or other shape such as a square or rectangular electrode, which projects a smaller distance (in relation to the length of the elongate electrode) from the distal end of IMD 80 or is flush with the distal end. The shorter electrode can project a fixed distance from the distal end of IMD 80, or it can be flexible or springlike in nature such that a force can push the shorter electrode closer to IMD 80, and the shorter electrode can return toward its original position upon reduction or removal of the force. Alternatively, partial electrode coil 82 may be substantially similar to second electrode 28 of FIGS. 1, 2, and 3. In some examples, partial electrode coil 82 includes one or more surfaces such as partial electrode coil surface(s) 84.

In this specification and in the drawings, there is presented and depicted a description of the functions, features, material composition of, and the application of a coating 94 to elongate electrode coil 86 and partial electrode coil 82. It should be understood that such description provided herein applies as well to any of the various forms or embodiments of the elongate electrode (in place of elongate electrode coil 86) and of the shorter electrode (in place of partial electrode coil 82) described herein, or to any alternatives may be apparent to a person of ordinary skill in the relevant art.

In some examples, IMD 80 may be implanted within a human patient. In alternative examples, IMD 80 may be implanted in non-human patients, such as primates, canines, equines, pigs, bovines, and felines. These other animals may undergo clinical or research therapies that may benefit from the subject matter of this disclosure.

In the example of FIG. 4, IMD 80 is a leadless pacing device, e.g., IMD 80 is not connected to any medical electrical lead and may be wholly implanted within a patient. In some examples, IMD 80 may include one or more leadless medical devices including one or more electrodes configured as described. In other examples, IMD 80 may include one or more medical devices, electrical leads, external devices, or other components that include medical electrical leads. IMD 80 may include, but is not limited to, medical devices such as an implantable cardioverter-defibrillator, neuromuscular stimulator devices, neurostimulator devices, pacing devices, or the like. In some examples, IMD 80 is generally cylindrical or pill-shaped and hermetically sealed to prevent ingress of fluid. In some examples, IMD 80 is an atrial pacing device configured to monitor ventricular events and control atrial pacing pulse delivery based on sensed ventricular events (or lack thereof) to promote atrial-ventricular synchrony for a patient. In some examples, IMD 80 is a ventricular pacing device configured to monitor electrical activity of a patient's heart and to control ventricular pacing pulse delivery based on sensed atrial events (or lack thereof). In some examples, IMD 80 is connected to at least one medical electrical lead. In some examples, IMD 80 is connected to two or more medical electrical leads.

Similar to first electrode 26 of FIGS. 1, 2, and 3, in some examples, elongate electrode coil 86 is configured to engage with cardiac tissue to deliver electrical stimulation therapy to a patient. In some examples, elongate electrode coil 86 is configured to sense electrical physiological signals. Elongate electrode coil 86 may include or be formed from any suitable electrically conductive material. In some examples, elongate electrode coil 86 may include a substrate (e.g., wire). In some examples, the elongate electrode coil 86 may comprise a platinum alloy that includes, but is not limited to, iridium. In some examples, elongate electrode coil 86 may include a single filar coil. In other examples, elongate electrode coil 86 may include a multi-filar coil, such as a bifilar coil. In some examples, elongate electrode coil 86 may include a sharpened distal tip.

IMD 80 may be configured to include housing 30 of FIG. 2. As illustrated in FIG. 4, IMD 80 may include elongate electrode coil 86 and partial electrode coil 82, which may be configured the same way as first electrode 26 and second electrode 28, respectively, of FIGS. 1, 2, and 3. For example, IMD 80 may be configured to include processing circuitry, electrical sensing circuitry, electrical pacing circuitry, and a power source, which may be configured as a rechargeable or non-rechargeable battery, communication circuitry, sensor circuitry, and a non-transitory memory to implement functionality attributable to a leadless pacemaker device, such as the Micra Transcatheter Pacing System from Medtronic Public Limited Company, of Fridley, Minnesota (operational headquarters).

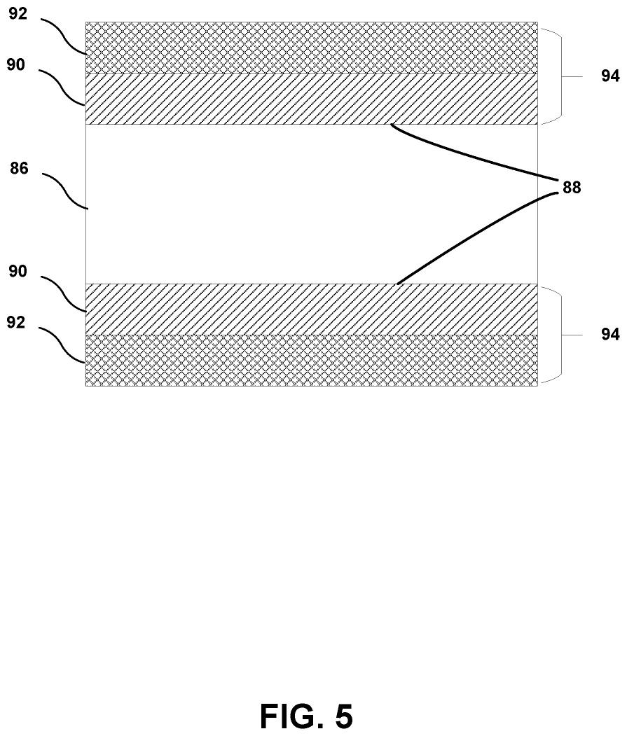

FIG. 5 is a conceptual cross-sectional diagram illustrating an example configuration of elongate electrode coil 86 of FIG. 4, which may be substantially similar to first electrode 26 of FIGS. 1, 2, and 3. In the illustrated example, elongate electrode coil 86 includes a coating 94 that is applied to one or more surfaces of elongate electrode coil 86, such as elongate electrode coil surface(s) 88. As shown in the example of FIG. 5, coating 94 comprises a first adhesive layer 90 comprising porous titanium nitride in contact with elongate electrode coil 86. Layer 90 may comprise other compounds containing titanium or any suitable other compound that provides a “fractal” surface, or a surface with columnar grains terminating in tips defined by crystallographic facets. In some examples, for example, first adhesive layer 90 comprising porous titanium nitride forms a fractal surface underlying a second electrically insulated layer comprising parylene. In the example of FIG. 5, elongate electrode coil 86 (or at least an outermost portion thereof) comprises, or is formed from, a platinum alloy. In some examples, elongate electrode coil 86 (or at least an outermost portion thereof) comprises, or is formed from, platinum and iridium, or a nickel-cobalt-chromium-molybdenum alloy, or a stainless-steel alloy such as 316L, or Nitinol, or any other suitable conductive and biocompatible materials.

Coating 94 further includes a second electrically insulated layer 92 comprising parylene in contact with first adhesive layer 90. As described herein, first adhesive layer 90 may be a porous titanium nitride layer applied to elongate electrode coil 86 to improve the adhesion of second electrically insulated layer 92 to elongate electrode coil 86. As described herein, second electrically insulated layer 92 may be a parylene layer. Parylene is a flexible biomedical coating for chronic implants such as the pacing electrode. Improvement in parylene adhesion may eliminate possible failures such as delamination, swelling, or tearing in vivo that could compromise implant biostability and function in the chronic phase, thus allowing for longer term stability and reliability of implants. In some examples, the thickness of first adhesive layer 90 is substantially uniform throughout the entirety of coating 94, e.g., the thickness of first adhesive layer 90 is substantially uniform over elongate electrode coil surface 88 of elongate electrode coil 86 of which coating 94 is applied. In some examples, the thickness of second electrically insulated layer 92 is substantially uniform throughout the entirety of coating 94, e.g., the thickness of second electrically insulated layer 92 is substantially uniform over elongate electrode coil surface 88 of which coating 94 is applied. In some examples, the thickness of coating 94 is substantially uniform over the entire surface of elongate electrode coil 86. In some examples, the most distal quarter turn or 90° of elongate electrode coil 86 only includes first adhesive layer 90, thus facilitating pacing only at the tip of elongate electrode coil 86. In these examples, silicone tubing is used to mask the most distal quarter turn portion of elongate electrode coil 86 that does not include second electrically insulated layer 92 while second electrically insulated layer 92 is applied. The silicone tubing may then be removed after the application of second electrically insulated layer 92. In some examples, the most distal quarter turn or 90° of elongate electrode coil 86 may not include first adhesive layer 90 and second electrically insulated layer 92.

In some examples, the thickness of first adhesive layer 90 varies along the distal tip of elongate electrode coil 86 or varies in other locations on the electrode(s) of device 10/IMD 80. For example, first adhesive layer 90 may be thickest on the ground, distal-facing surface of the tip of elongate electrode coil 86 and thinnest on the opposing side of elongate electrode coil 86; i.e., first adhesive layer 90 may be thickest on distal-facing aspects of elongate electrode coil 86.

In some examples, coating 94 is also applied to one or more surfaces of partial electrode coil 82, such as partial electrode coil surface(s) 84 of FIG. 4. The thickness of second electrically insulated layer 92 may be substantially uniform over partial electrode coil surface 84 of which coating 94 is applied. In some examples, the thickness of second electrically insulated layer 92 may be substantially uniform over the entire surface of partial electrode coil 82.

First adhesive layer 90 may be applied by first depositing a porous titanium nitride coating with a thickness ranging from 100 nanometers to 5 micrometers onto a helical platinum alloy (e.g., platinum/iridium) electrode such as elongate electrode coil 86. First adhesive layer 90 may then serve as a 3D adhesion promoter layer on elongate electrode coil 86. Second electrically insulated layer 92 may then be deposited and bonded to the 3D first adhesive layer 90 to form adhesion enhanced parylene with a thickness ranging from 100 nanometers to 10 micrometers. In some examples, a plasma cleaning process is applied after first adhesive layer 90 is applied and before second electrically insulated layer 92 is applied. The plasma cleaning process may use a gas plasma to remove contaminants and impurities, e.g., dirt, dust, oil, and organic material, from elongate electrode coil surface 88 and first adhesive layer 90 without causing damage to elongate electrode coil surface 88 and first adhesive layer 90. Further, the plasma cleaning process may aid in removing surface contaminants that are often difficult to remove using other methods, such as ionic contamination or hydrocarbons.

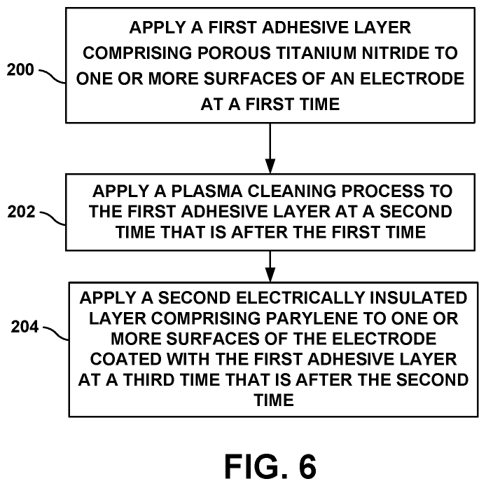

FIG. 6 is a flow diagram illustrating an example technique for forming an implantable medical device including a coated electrode. The technique includes applying a first adhesive layer comprising porous titanium nitride to one or more surfaces of an electrode at a first time (200). The first adhesive layer may be substantially similar to first adhesive layer 90 of FIG. 5. The electrode may be substantially similar to first electrode 26 of FIGS. 1, 2, and 3 or elongate electrode coil 86 of FIG. 4. The one or more surfaces of the electrode may be substantially similar to elongate electrode coil surface 88 of FIG. 4. The technique further includes applying a plasma cleaning process to the first adhesive layer at a second time that is after the first time (202). The technique further includes applying a second electrically insulated layer comprising parylene to the one or more surfaces of the electrode coated with the first adhesive layer at a third time that is after the second time (204). The second electrically insulated layer may be substantially similar to second electrically insulated layer 92 of FIG. 5. The second electrically insulated layer may be applied with a uniform thickness throughout the entirety of the coating.

Various examples have been described. This disclosure includes the following non-limiting examples.
Example 1. An implantable medical device comprising: an electrode; a power source; electrical pacing circuitry that is coupled to the power source and that is configured to deliver cardiac pacing therapy; and a coating applied to one or more surfaces of the electrode, wherein the coating comprises a first adhesive layer comprising porous titanium nitride and a second electrically insulated layer comprising parylene, and wherein a plasma cleaning process is applied to the first adhesive layer prior to the second electrically insulated layer.
Example 2. The implantable medical device of example 1, wherein a thickness of the second electrically insulated layer is substantially uniform throughout the entirety of the coating.
Example 3. The implantable medical device of example 1 or 2, wherein the electrode is an elongate electrode configured to extend distally from a distal portion of the implantable medical device.
Example 4. The implantable medical device of example 1 or 3, wherein the electrode comprises a first electrode, and the implantable medical device further comprises further comprising a second electrode located on the distal portion of the implantable medical device, wherein a length of the second electrode is less than a length of the elongate electrode, and wherein the second electrode one of extends a smaller distance from the distal portion of the implantable medical device than the elongate electrode or is flush with the distal portion of the implantable medical device.
Example 5. The implantable medical device of examples 1-4, wherein the first adhesive layer comprising porous titanium nitride is applied to the one or more surfaces of the electrode at a first time, wherein the plasma cleaning process is applied to the first adhesive layer at a second time that is after the first time, and wherein the second electrically insulated layer comprising parylene is applied to the one or more surfaces of the electrode coated with the first adhesive layer at a third time that is after the second time.
Example 6. The implantable medical device of examples 1-5, wherein the first adhesive layer comprising porous titanium nitride forms a fractal surface underlying the second electrically insulated layer comprising parylene.
Example 7. The implantable medical device of any one or more of examples 1-6, wherein the implantable medical device is configured as a leadless pacing device comprising a housing for the power source and the electrical pacing circuitry, and wherein the electrode extends from the housing.
Example 8. The implantable medical device of examples 1-7, wherein the electrode comprises an alloy including platinum and iridium.
Example 9. The implantable medical device of examples 1-8, wherein the electrode is configured as a coil comprising a plurality of turns of at least one filar.
Example 10. The implantable medical device of example 9, wherein the coil forms a helix.
Example 11. The implantable medical device of examples 1-10, wherein the first adhesive layer comprising porous titanium nitride comprises a thickness ranging from 100 nanometers to 5 micrometers.
Example 12. The implantable medical device of examples 1-11, wherein the second electrically insulated layer comprising parylene comprises a thickness ranging from 100 nanometers to 10 micrometers.
Example 13. The implantable medical device of examples 1-12, wherein the first adhesive layer comprising porous titanium nitride is thermally bonded to the second electrically insulated layer comprising parylene.
Example 14. A method for forming an implantable medical device that includes an electrode, a power source, electrical pacing circuitry that is coupled to the power source and that is configured to deliver cardiac pacing therapy, and a coating applied to one or more surfaces of the electrode, wherein the coating comprises a first adhesive layer comprising porous titanium nitride and a second electrically insulated layer comprising parylene, and wherein a plasma cleaning process is applied to the first adhesive layer prior to the second electrically insulated layer, the method comprising: applying the first adhesive layer comprising porous titanium nitride to the one or more surfaces of the electrode at a first time; applying the plasma cleaning process to the first adhesive layer at a second time that is after the first time; and applying the second electrically insulated layer comprising parylene to the one or more surfaces of the electrode coated with the first adhesive layer at a third time that is after the second time.
Example 15. The method of example 14, wherein a thickness of the second electrically insulated layer is substantially uniform throughout the entirety of the coating.
Example 16. The method of example 14 or 15, wherein the electrode is an elongate electrode configured to extend distally from a distal portion of the implantable medical device.
Example 17. The method of example 14-16, wherein the electrode comprises a first electrode, and the implantable medical device further comprises further comprising a second electrode located on the distal portion of the implantable medical device, wherein a length of the second electrode is less than a length of the elongate electrode, and wherein the second electrode one of extends a smaller distance from the distal portion of the implantable medical device than the elongate electrode or is flush with the distal portion of the implantable medical device.
Example 18. The method of examples 14-17, wherein the first adhesive layer comprising porous titanium nitride forms a fractal surface underlying the second electrically insulated layer comprising parylene.
Example 19. The method of examples 14-18, wherein the implantable medical device is configured as a leadless pacing device comprising a housing for the power source and the electrical pacing circuitry, and wherein the electrode extends from the housing.
Example 20. The method of examples 14-19, wherein the electrode comprises an alloy including platinum and iridium.
Example 21. The method of examples 14-20, wherein the electrode is configured as a coil comprising a plurality of turns of at least one filar.
Example 22. The method of example 21, wherein the coil forms a helix.
Example 23. The method of examples 14-22, wherein the first adhesive layer comprising porous titanium nitride comprises a thickness ranging from 100 nanometers to 5 micrometers.
Example 24. The method of examples 14-23, wherein the second electrically insulated layer comprising parylene comprises a thickness ranging from 100 nanometers to 10 micrometers.
Example 25. The method of examples 14-24, wherein the first adhesive layer comprising porous titanium nitride is thermally bonded to the second electrically insulated layer comprising parylene.
Example 26. The method of examples 14-25, wherein applying the second electrically insulated layer comprises applying the second electrically insulated layer with a uniform thickness throughout the entirety of the coating.
Example 27. An implantable medical device comprising: a power source; electrical pacing circuitry that is coupled to the power source and that is configured to deliver cardiac pacing therapy; a first electrode that is configured to extend distally from a distal portion of the implantable medical device; a second electrode that is located on the distal portion of the implantable medical device; and a coating, wherein the coating comprises a first adhesive layer comprising porous titanium nitride and a second electrically insulated layer comprising parylene, and wherein a plasma cleaning process is applied to the first adhesive layer prior to the second electrically insulated layer.
Example 28. The implantable medical device of example 27, wherein the coating is applied to one or more surfaces of the first electrode and the second electrode.
Example 29. The implantable medical device of example 27 or 28, wherein a thickness of the second electrically insulated layer is substantially uniform throughout the entirety of the coating.
Example 30. The implantable medical device of examples 27-29, wherein the first electrode is an elongate electrode.
Example 31. The implantable medical device of examples 27-30, wherein a length of the second electrode is less than a length of the elongate electrode, and wherein the second electrode one of extends a smaller distance from the distal portion of the implantable medical device than the elongate electrode or is flush with the distal portion of the implantable medical device.
Example 32. The implantable medical device of examples 27-31, wherein the first adhesive layer comprising porous titanium nitride is applied to the one or more surfaces of the first electrode at a first time, wherein the plasma cleaning process is applied to the first adhesive layer at a second time that is after the first time, and wherein the second electrically insulated layer comprising parylene is applied to the one or more surfaces of the first electrode coated with the first adhesive layer at a third time that is after the second time.
Example 33. The implantable medical device of examples 27-32, wherein the first adhesive layer comprising porous titanium nitride forms a fractal surface underlying the second electrically insulated layer comprising parylene.
Example 34. The implantable medical device of examples 27-33, wherein the first electrode is configured as a coil comprising a plurality of turns of at least one filar.
Example 35. The implantable medical device of example 34, wherein the coil forms a helix.
Example 36. The implantable medical device of examples 27-35, wherein the first adhesive layer comprising porous titanium nitride comprises a thickness ranging from 100 nanometers to 5 micrometers.
Example 37. The implantable medical device of examples 27-36, wherein the second electrically insulated layer comprising parylene comprises a thickness ranging from 100 nanometers to 10 micrometers.
Example 38. The implantable medical device of examples 27-37, wherein the first electrode is configured to extend from the distal portion of the implantable medical device and penetrate through the wall tissue of a first heart chamber into the wall tissue of a second heart chamber.
Example 39. The implantable medical device of examples 27-38, wherein the second electrode is configured to extend from the distal portion of the implantable medical device and flexibly maintain contact with the wall tissue of the first heart chamber without penetration of the wall tissue of the first heart chamber.
Example 40. The implantable medical device of examples 27-39, wherein the thickness of the second electrically insulated layer is substantially uniform throughout the entirety of the coating.

1-15. (canceled)

16. An implantable medical device comprising:
an electrode;
a power source;
electrical pacing circuitry that is coupled to the power source and that is configured to deliver cardiac pacing therapy; and
a coating applied to one or more surfaces of the electrode, wherein the coating comprises a first adhesive layer comprising porous titanium nitride and a second electrically insulated layer comprising parylene, and wherein a plasma cleaning process is applied to the first adhesive layer prior to the second electrically insulated layer.

17. The implantable medical device of claim 16, wherein a thickness of the second electrically insulated layer is substantially uniform throughout the entirety of the coating.

18. The implantable medical device of claim 16, wherein the electrode is an elongate electrode configured to extend distally from a distal portion of the implantable medical device.

19. The implantable medical device of claim 18, wherein the electrode comprises a first electrode, wherein the implantable medical device further comprises a second electrode located on the distal portion of the implantable medical device, wherein a length of the second electrode is less than a length of the elongate electrode, and wherein the second electrode extends a smaller distance from the distal portion of the implantable medical device than the elongate electrode or is flush with the distal portion of the implantable medical device.

20. The implantable medical device of claim 16, wherein the first adhesive layer comprising porous titanium nitride is applied to the one or more surfaces of the electrode at a first time, wherein a plasma cleaning process is applied to the first adhesive layer at a second time that is after the first time, and wherein the second electrically insulated layer comprising parylene is applied to the one or more surfaces of the electrode coated with the first adhesive layer at a third time that is after the second time.

21. The implantable medical device of claim 16, wherein the first adhesive layer comprising porous titanium nitride forms a fractal surface underlying the second electrically insulated layer comprising parylene.

22. The implantable medical device of claim 16, wherein the implantable medical device is configured as a leadless pacing device comprising a housing for the power source and the electrical pacing circuitry, and wherein the electrode extends from the housing.

23. The implantable medical device of claim 16, wherein the electrode comprises an alloy including platinum and iridium.

24. The implantable medical device of claim 16, wherein the electrode is configured as a coil comprising a plurality of turns of at least one filar.

25. The implantable medical device of claim 24, wherein the coil forms a helix.

26. The implantable medical device of claim 16, wherein the first adhesive layer comprising porous titanium nitride comprises a thickness ranging from 100 nanometers to 5 micrometers.

27. The implantable medical device of claim 16, wherein the second electrically insulated layer comprising parylene comprises a thickness ranging from 100 nanometers to 10 micrometers.

28. The implantable medical device of claim 16, wherein the first adhesive layer comprising porous titanium nitride is thermally bonded to the second electrically insulated layer comprising parylene.

29. A method for forming an implantable medical device that includes an electrode, a power source, electrical pacing circuitry that is coupled to the power source and that is configured to deliver cardiac pacing therapy, and a coating applied to one or more surfaces of the electrode, wherein the coating comprises a first adhesive layer comprising porous titanium nitride and a second electrically insulated layer comprising parylene, and wherein a plasma cleaning process is applied to the first adhesive layer prior to the second electrically insulated layer, the method comprising:
applying the first adhesive layer comprising porous titanium nitride to the one or more surfaces of the electrode at a first time;
applying the plasma cleaning process to the first adhesive layer at a second time that is after the first time; and
applying the second electrically insulated layer comprising parylene to the one or more surfaces of the electrode coated with the first adhesive layer at a third time that is after the second time.

30. The method of claim 29, wherein a thickness of the second electrically insulated layer is substantially uniform throughout the entirety of the coating.

31. The method of claim 29, wherein the first adhesive layer comprising porous titanium nitride forms a fractal surface underlying the second electrically insulated layer comprising parylene.

32. The method of claim 29, wherein the electrode is configured as a coil comprising a plurality of turns of at least one filar.

33. The method of claim 29, wherein the first adhesive layer comprising porous titanium nitride comprises a thickness ranging from 100 nanometers to 5 micrometers.

34. The method of claim 29, wherein the second electrically insulated layer comprising parylene comprises a thickness ranging from 100 nanometers to 10 micrometers.

35. The method of claim 29, wherein the first adhesive layer comprising porous titanium nitride is thermally bonded to the second electrically insulated layer comprising parylene.
